(19) United States
(12) Patent Application Publication
(10) Pub. No.: US 2010/0249725 A1
(43) Pub. Date: Sep. 30, 2010

(54) Medical Valve with Multiple Variable Volume Regions

(75) Inventors: Andrew L. Cote, SR., Merrimack, NH (US); Jake P. Ganem, Cape Neddick, ME (US)

Correspondence Address: Sunstein Kann Murphy & Timbers LLP, 125 SUMMER STREET, BOSTON, MA, 02110-1618, US

(73) Assignee: NP MEDICAL INC., Clinton, MA (US)

(21) Appl. No.: 12/750,120

(22) Filed: Mar. 30, 2010

Related U.S. Application Data
(60) Provisional application No. 61/164,585, filed on Mar. 30, 2009.

(51) Int. Cl. A61M 5/00

(52) U.S. Cl. 604/249

(57) ABSTRACT

A medical valve transitions between an open mode that permits fluid flow, and a closed mode that prevents fluid flow. The valve has a housing, a valve member with a distal seal having a normally closed aperture, a first variable volume region and a second variable volume region longitudinally spaced from the first. Insertion of a medical implement into the inlet opens the aperture and fluidly connects the valve inlet and outlet via a fluid path. Withdrawal of the implement closes the aperture and fluidly disconnects the inlet and outlet. The first variable volume region contracts and the second variable volume expands upon withdrawal of the implement. Both variable volumes may be part of the fluid path. The fluid path may have a closed volume before insertion of the implement and an open volume when in an open mode. The closed volume may be substantially equal to the open mode.

### PRIORITY

This patent application claims priority from U.S. Provisional Patent Application No. 61/164,585, filed Mar. 30, 2009, entitled, “Medical Valve with Distal Seal Actuator,” and naming Andy L. Cote and Jake Ganem as inventors, the disclosure of which is incorporated herein, in its entirety, by reference.

### RELATED UNITED STATES PATENT APPLICATIONS

This patent application is related to the following co-pending U.S. patent application:

U.S. patent application Ser. No. ______, entitled, “MEDICAL VALVE WITH DISTAL SEAL ACTUATOR,” naming Andrew L. Cote and Jake P. Ganem as inventors, filed on even date herewith, and assigned attorney docket number 1600/A08, the disclosure of which is incorporated herein, in its entirety, by reference.

### FIELD OF THE INVENTION

The invention generally relates to medical valves and, more particularly, the invention relates to mitigating fluid drawback through medical valves.

### BACKGROUND

In general terms, medical valving devices often act as a sealed port that may be repeatedly accessed to non-invasively inject fluid into (or withdraw fluid from) a patient's vasculature. Consequently, a medical valve permits the patient's vasculature to be freely accessed without requiring such patient's skin be repeatedly pierced by a needle.

Medical personnel insert a medical instrument into the medical valve to inject fluid into (or withdraw fluid from) a patient who has an appropriately secured medical valve. Once inserted, fluid may be freely injected into or withdrawn from the patient. Problems can arise, however, when the medical instrument is withdrawn from the valve. Specifically, suction produced by the withdrawing medical instrument can undesirably cause blood to be drawn proximally into or toward the valve. In addition to coagulating and impeding the mechanical operation of the valve, blood in the valve also compromises the sterility of the valve.

### SUMMARY OF THE INVENTION

In accordance with one embodiment of the present invention, a medical valve transitions between an open mode that permits fluid flow, and a closed mode that prevents fluid flow. The medical valve may include a housing with an inlet and an outlet, a valve member within the housing, a first variable volume region and a second variable volume region. The valve member may have a distal seal member with a normally closed aperture passing through it. The distal seal may include a protrusion that extends distally from the distal seal and provides a dynamic back pressure seal. The normally closed aperture may pass through the protrusion and insertion of a medical implement into the inlet may open the aperture and fluidly connect the inlet and outlet via a fluid path between the inlet and outlet. Conversely, withdrawal of the medical implement may close the aperture and fluidly disconnect the inlet and outlet.

The first and second variable volume regions may be longitudinally spaced from one another and may be part of the fluid path. The first variable volume region may contract and the second variable volume may expand upon withdrawal of the medical implement. Conversely, the first variable volume may expand and the second variable volume may contract upon insertion of the medical instrument. The fluid path may have a closed volume before insertion of the medical implement and an open volume in the open mode. The closed volume may be substantially equal to the open volume. Additionally, the volumes of the first and second variable volume regions may be configured, respectively, to contract and expand to produce substantially neutral fluid displacement at the outlet during disconnection and/or connection of the medical implement.

The open volume of the fluid path may be the volume of the fluid path when the medical implement is inserted to its farthest point. Alternatively, the open volume of the fluid path may be the volume of the fluid path when the medical implement is only partially inserted, when the aperture first opens, or when the aperture is fully open.

In accordance with some embodiments, the distal seal member may bound the second variable volume region when the valve is in the closed mode, and the valve may include a post member that is moveably mounted within the housing. The post member may move distally within the housing to fluidly connect the inlet and outlet upon insertion of the medical implement into the inlet. The post member may move proximally within the housing to fluidly disconnect the inlet and outlet upon withdrawal of the medical implement. The post member may have a flow channel that fluidly communicates the first volume and the second volume when the valve is in the open mode. The flow channel may also allow fluid from the first volume to enter the second volume as the first volume contracts.

The post member may include a tube portion and a head portion (e.g., a post head) that protrudes radially outward from the tube portion. The head portion may include a proximally facing surface, a distally facing surface, and at least one post head channel extending between the proximally facing surface and the distally facing surface. Additionally, the first variable volume may have a first sub-volume and a second sub-volume that are separated by the head portion. For example, the first sub-volume may be proximal to the proximally facing surface and the second sub-volume may be distal to the distally facing surface. Upon withdrawal of the medical implement, the first sub-volume may contract and the second sub-volume may expand. The post head channel fluidly connects the first and second sub-volumes.

In accordance with further embodiments, the head portion may have a plurality of distally extending protrusions that apply a force on the distal seal to open the aperture (e.g., as the medical implement is inserted). Alternatively, the post member may include a tube portion and a plurality of legs extending distally from a distal end of the tube portion. The leg portions may apply a force on the distal seal to open the aperture as the medical implement is inserted into the inlet. For example, the plurality of legs may apply a radially directed force and a longitudinally directed force on the distal seal as the post member moves distally. The radially directed force and longitudinally directed force may open the aperture and deform the distal seal member.

In accordance with other embodiments, at least a portion of the distal seal member may invert from a first shape (e.g., concave) to a second shape (e.g., convex) as the aperture opens. Additionally, the valve may also include a proximal seal member with a normally closed aperture through it. In such embodiments, insertion of the medical implement moves the proximal seal member distally and opens the proximal seal aperture. The proximal seal member may be swabbable when the valve is in the closed mode. The first variable volume may be located between the post member and the distal seal and the second variable volume may be located between the post member and the proximal seal.

In accordance with some embodiments, the medical implement may travel a distal stroke distance to open the aperture and a proximal stroke distance to close the aperture. The distal stroke distance may be the distance from initial connection of the medical implement to the point at which the aperture first opens. The proximal stroke distance may be the distance from the point at which the medical implement is fully inserted to the point at which the aperture first closes. The proximal stroke distance may be less than the distal stroke distance (e.g., 25% of the distal stroke distance).

In accordance with additional embodiments of the present invention, a method connects a medical valve to a patient. Among other things, the medical valve may include a housing with an inlet and an outlet, a valve member with a distal seal member having a normally closed aperture, a first variable volume region, and a second variable volume region. The first variable volume region may be longitudinally spaced from the second variable volume region. The method then inserts a medical implement through the inlet and moves the medical implement distally within the housing. The distal movement of the medical implement transitions the valve from a closed mode to an open mode by opening the normally closed aperture and fluidly connecting the inlet and outlet via a fluid path between the inlet and the outlet. The first and second variable volumes may be part of the fluid path.

The method may then transfer fluid between the medical implement and the patient through the valve. After transferring fluid, the method may move the medical implement proximally within the housing to fluidly disconnect the inlet and outlet by closing the aperture. During withdrawal of the medical implement, the first variable volume region may contract and the second variable volume region may expand. The fluid path may have a closed volume before insertion of the medical implement and an open volume when in the open mode. The closed volume may be substantially equal to the open volume.

In accordance with further embodiments of the present invention, a medical valve transitions between an open mode that permits fluid flow, and a closed mode that prevents fluid flow. The medical valve may include a housing with an inlet and an outlet, a valve means in the housing, a first means for forming a first variable volume, and a second means for forming a second variable volume. The valve means may have a distal sealing means with a normally closed aperture. Insertion of a medical implement into the inlet may open the aperture and fluidly connect the inlet and outlet via a fluid path between the inlet and outlet. Withdrawal of the medical implement may close the aperture and fluidly disconnect the inlet and outlet.

The second variable volume region may be longitudinally spaced from the first variable volume region. The first variable volume region may contract and the second variable volume region may expand upon withdrawal of the medical implement. The fluid path may have a closed volume before insertion of the medical implement and an open volume when in the open mode. The closed volume may be substantially equal to the open volume.

In accordance with further embodiments, a medical valve has an open mode that permits fluid flow, and a closed mode that prevents fluid flow. The medical valve may also include a housing with an inlet and an outlet, a valve member within the housing, a first variable volume region, and a second variable volume region. The valve member may have a distal seal member with a normally closed aperture therethrough. Insertion of a medical implement into the inlet may open the aperture and fluidly connect the inlet and outlet via a fluid path between the inlet and the outlet. Withdrawal of the medical implement closes the aperture and fluidly disconnects the inlet and outlet.

The second variable volume region may be longitudinally spaced from the first variable volume region. The first and second variable volume regions may be part of the fluid path. The first variable volume region may contract upon withdrawal of the medical implement and the second variable volume region may expand upon withdrawal of the medical implement.

The fluid path may have a closed volume before insertion of the medical implement and an open volume when in the open mode. The closed volume may be less than the open volume. The volumes of the first and second variable volume regions may be configured to respectively contract and expand to produce a positive fluid displacement at the outlet during disconnection of the medical implement.

### BRIEF DESCRIPTION OF THE DRAWINGS

The foregoing features of the invention will be more readily understood by reference to the following detailed description, taken with reference to the accompanying drawings, in which:

FIG. 1 schematically shows one use of a medical valve configured in accordance with one embodiment of the present invention.

FIG. 2A schematically shows a perspective view of a medical valve configured in accordance with illustrative embodiments of the present invention.

FIG. 2B schematically shows a perspective view of a medical valve of FIG. 2A with a Y-site branch.

FIG. 3 schematically shows a cross-sectional view of the valve shown in FIG. 2A in the closed mode along line 3-3.

FIG. 4 schematically shows a cross-sectional view of the valve shown in FIG. 2A in the open mode along line 3-3.

FIG. 5 schematically shows a perspective view of an illustrative embodiment of a resilient member within the valve of FIG. 2A.

FIG. 6 schematically shows a perspective view of an illustrative embodiment of a moveable plug member within the valve of FIG. 2A.

FIG. 7 schematically shows a perspective view of an alternative embodiment of a moveable plug member within the valve of FIG. 2A.

FIG. 8 shows a process of using the medical valve shown in FIG. 2A in accordance with illustrative embodiments of the invention.

FIG. 9 schematically shows a perspective view of an illustrative embodiment of an alternative moveable plug member, in accordance with additional embodiments of the present invention.

FIG. 10 schematically shows a cross-sectional view of an alternative embodiment of a medical valve having the post member with leg members shown in FIG. 9. This figure shows the valve in the closed mode.

FIG. 11 schematically shows a cross-sectional view of the medical valve shown in FIG. 10 in the open mode.

### DESCRIPTION OF ILLUSTRATIVE EMBODIMENTS

In illustrative embodiments, a medical valve has an internal valve mechanism with a post member that is moveable to open an aperture in a resilient member. The medical valve may also have multiple variable volume regions and a quick close aperture so that the valve has a substantially neutral fluid displacement at the outlet upon connection and/or disconnection of a medical instrument. Details of illustrative embodiments are discussed below.

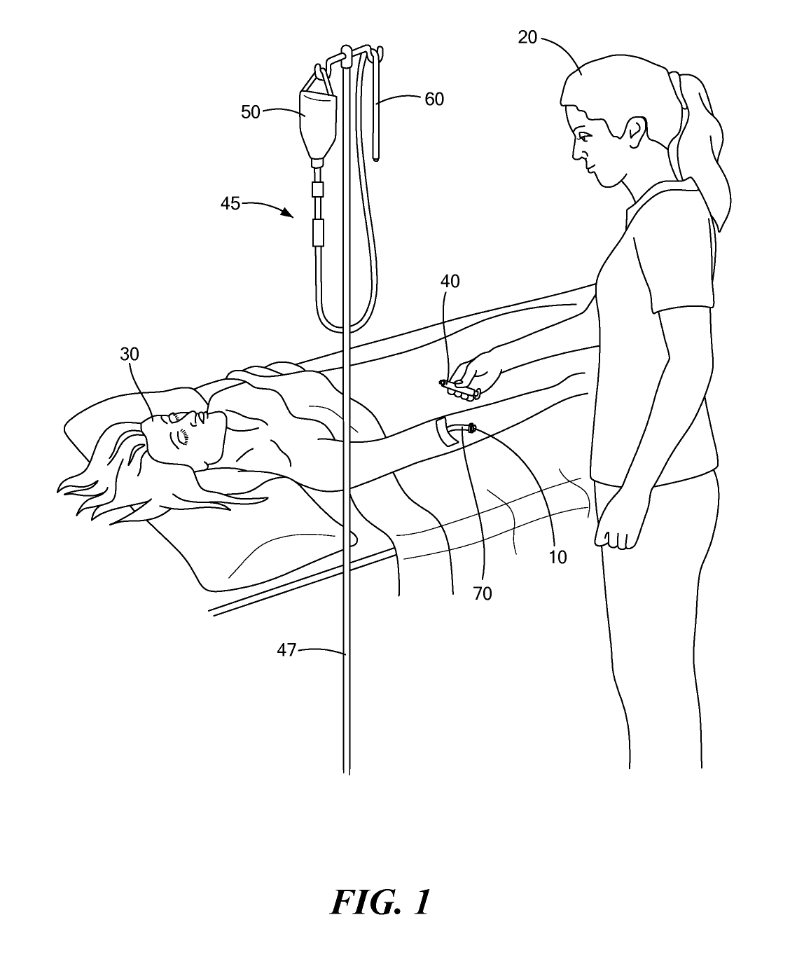

FIG. 1 schematically shows one illustrative use of a medical valve 10 configured in accordance with illustrative embodiments of the invention. In this example, a catheter 70 connects the valve 10 with a patient's vein (the patient is identified by reference number 30). Adhesive tape or similar material may be coupled with the catheter 70 and patient's arm to ensure that the valve 10 remains in place.

After the valve 10 is in place, a nurse, doctor, technician, practitioner, or other user (schematically identified by reference number 20) may intravenously deliver medication to the patient 30, who is lying in a hospital bed. To that end, before the valve 10 is properly primed and flushed (e.g., with a saline flush), the nurse 20 swabs the top surface of the valve 10 to remove contaminants. Next, the nurse 20, once again, swabs the top surface of the valve 10 and uses a medical instrument 40 (e.g., a syringe having a distally located blunt, luer tip complying with ANSI/ISO standards) to inject medication into the patient 30 through the valve 10. For example, the medical practitioner 20 may use the valve 10 to inject drugs such as heparin, antibiotic, pain medication, other intravenous medication, or other fluid deemed medically appropriate. Alternatively, the nurse 20 (or other user) may withdraw blood from the patient 30 through the valve 10.

The medical valve 10 may receive medication or other fluids from other means, such as through a gravity feed system 45. In general, traditional gravity feeding systems 45 often have a bag 50 (or bottle) containing a fluid (e.g., anesthesia medication) to be introduced into the patient 30. The bag 50 (or bottle) typically hangs from a pole 47 to allow for gravity feeding. The medical practitioner 20 then connects the bag/bottle 50 to the medical valve 10 using tubing 60 having an attached blunt tip. In illustrative embodiments, the blunt tip of the tubing has a luer taper that complies with the ANSI/ISO standard.

After the tubing 60 is connected to the medical valve 10, gravity (or a pump) causes the fluid to begin flowing into the patient 30. In some embodiments, the feeding system 45 may include additional shut-off valves on the tubing 60 (e.g., stop-cock valves or clamps) to stop fluid flow without having to disconnect the tubing 60 from the valve 10. Accordingly, the valve 10 can be used in long-term “indwell” procedures.

After administering or withdrawing fluid from the patient 30, the nurse 20 should appropriately swab and flush the valve 10 and catheter 70 to remove contaminants and ensure proper operation. As known by those skilled in the art, there is a generally accepted valve swabbing and flushing protocol that should mitigate the likelihood of infection. Among other things, as summarized above, this protocol requires proper flushing and swabbing before and after the valve 10 is used to deliver fluid to, or withdraw fluid from the patient 30.

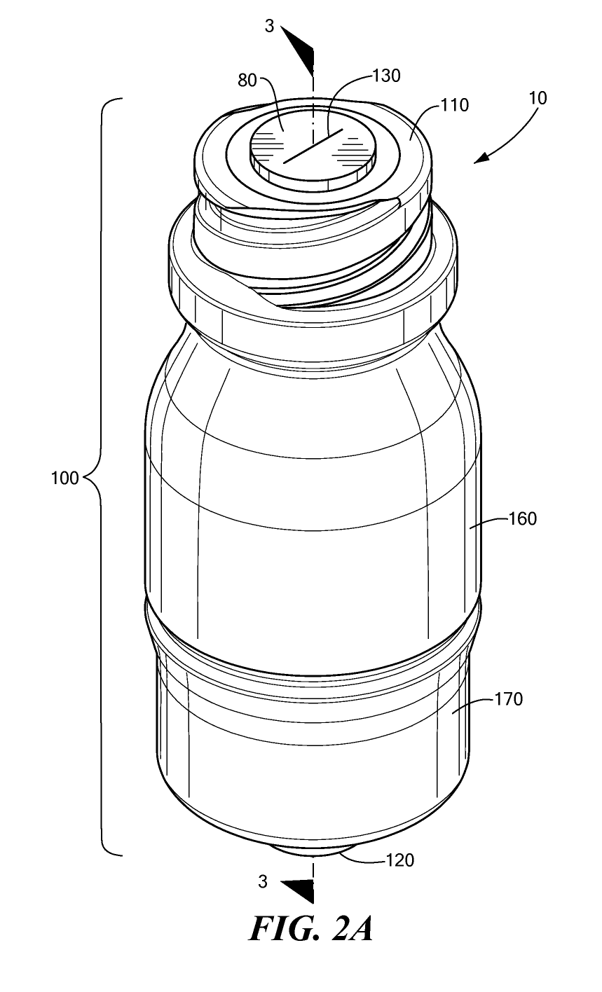

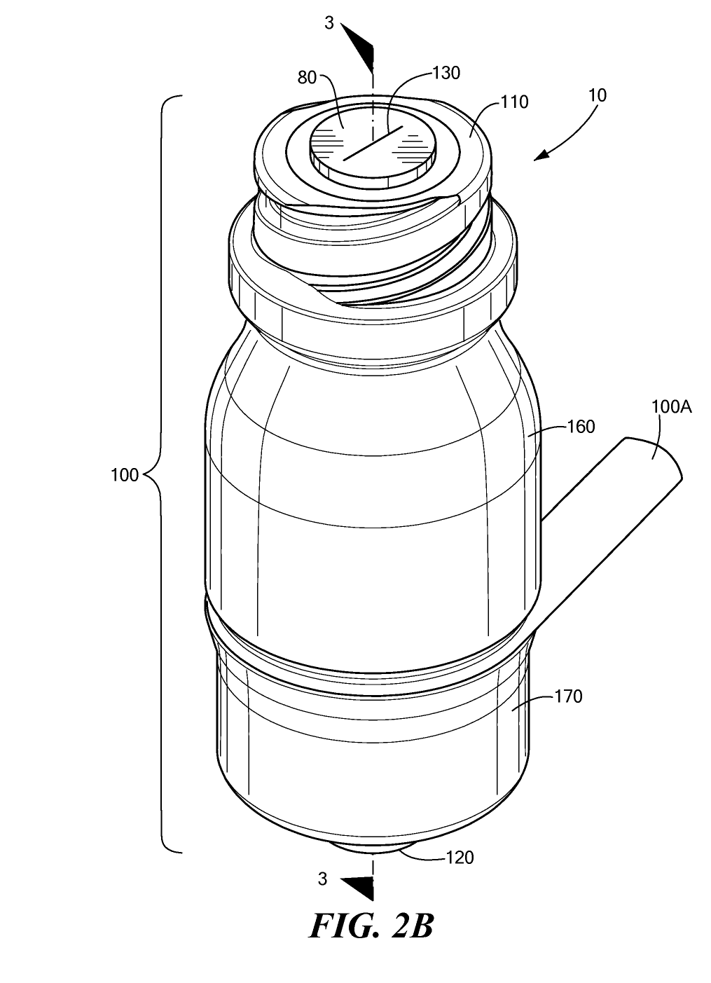

FIG. 2A schematically shows a perspective view of the medical valve 10 shown in FIG. 1, while FIG. 2B schematically shows the same valve with a Y-site branch 100A. In illustrative embodiments, during withdrawal of the instrument 40, the valve 10 may be configured to have a substantially positive fluid displacement or a substantially neutral fluid displacement (between about plus or minus 1 microliter of fluid displacement, discussed below). In other words, withdrawal of a medical instrument 40 causes either a positive fluid displacement or essentially no or negligible fluid displacement at the distal end of the valve 10.

In this context, fluid displacement generally refers to the flow of fluid through the distal port 120 of the valve 10 (discussed below). Accordingly, a positive fluid displacement generally refers to fluid flowing in a distal direction through the distal port 120, while a negative fluid displacement generally refers to a fluid flowing in a proximal direction through the distal port 120. Of course, not all embodiments exhibit this quality. For example, in alternative embodiments, the valve 10 may have a negative fluid displacement when the instrument 40 is withdrawn.

It should be noted that the fluid displacements discussed herein refer to the “net” fluid displaced through the distal port 120. Specifically, during insertion or withdrawal of the instrument 40, the actual flow of fluid through the distal port 120 may change direction and thus, fluctuate. However, when considering this fluctuation, the net change in fluid flow through the distal port 120 should be 1) positive when the valve exhibits a “positive fluid displacement,” and 2) negative when the valve exhibits a “negative fluid displacement.” In a similar manner, a substantially neutral fluid displacement occurs when, as noted above, the valve 10 has a net fluid displacement of between about plus or minus one microliter. Of course, the fluid displacement of the valve 10 is discussed herein in terms of one stroke of the instrument 40 (i.e., insertion or withdrawal of the instrument 40).

Ideally, a valve with a neutral displacement has 0.0 microliters of positive or negative fluid displacement. As suggested above, however, in practice, a neutral displacement actually can have a very slight positive or negative displacement (e.g., caused by a manufacturing tolerance), such as a displacement on the order of positive or negative one microliter, or less. In other words, in such embodiments, the volumes of fluid forced through the distal port 120 in a neutral displacement valve are negligible (ideally zero microliters) and should have a negligible impact on the goals of the valve.

Some embodiments may have a very low positive or negative fluid displacement upon withdrawal. For example, such valves may have a negative fluid displacement of about one to two microliters (i.e., about one to two microliters of fluid drawback, which is proximally directed), or about one to two microliters positive fluid displacement (i.e., about one to two microliters of positively pushed fluid, which is distally directed). Although such amounts are in the positive or negative fluid displacement ranges, they still should represent a significant improvement over valves that exhibit higher positive or negative fluid displacements upon withdrawal.

The neutral, positive, or negative fluid displacement of a valve may be corrupted by manual handling of the valve 10, catheter 70 or the instrument 40 during the fluid transfer. For example, a slight inward force applied to the shaft of the medical instrument 40 (e.g., by the nurse's hand when simply holding the medical instrument 40) can have the effect of adding a positive fluid displacement from the medical instrument 40 (when the force is applied) and, ultimately, through the valve 10. In fact, releasing this force from the medical instrument 40 actually may draw fluid proximally, causing a negative fluid displacement that further corrupts fluid displacement. These effects, however, should not be considered when determining the nature of fluid displacement through the distal port 120. To overcome the problem noted above with regard to squeezing the medical instrument shaft, for example, the nurse 20 can hold another part of the medical instrument that does not contain the fluid (e.g., stubs at the proximal end of the medical instrument 40).

To accomplish these desired goals, the valve 10 has a housing 100 forming an interior having a proximal port 110 for receiving the instrument 40, and the noted distal port 120 having the discussed fluid displacement properties. The valve 10 has an open mode that permits fluid flow through the valve 10, and a closed mode that prevents fluid flow through the valve 10. To that end, the interior contains a valve mechanism that selectively controls (i.e., allow/permits) fluid flow through the valve 10. The fluid passes through a complete fluid path that extends between the proximal port 110 and the distal port 120.

It should be noted that although much of the discussion herein refers to the proximal port 110 as an inlet, and the distal port 120 as an outlet, the proximal and distal ports 110 and 120 also may be respectively used as outlet and inlet ports. Discussion of these ports in either configuration therefore is for illustrative purposes only.

The valve 10 is considered to provide a low pressure seal at its proximal end 110. To that end, the proximal end 110 of the medical valve 10 has a resilient proximal seal 80 with a resealable aperture 130 that extends entirely through its profile. The aperture 130 may, for example, be a pierced hole or a slit. Alternatively, the proximal seal 80 may be molded with the aperture 130. To help center the proximal seal 80 within the proximal port 110 and keep the aperture 130 closed (e.g., by pre-loading the aperture 130), the proximal gland may have centering ribs 82 nearer the proximal end of the proximal seal 80.

As mentioned above, some embodiments of the present invention may be swabbable. To that end, the proximal seal 80 may be substantially flush with or extend slightly proximal to the proximal port 110 when the valve 10 is in the closed mode. This creates a swabbable surface at the inlet of the valve 10 and allows the nurse 20 to perform the swabbing protocol discussed above.

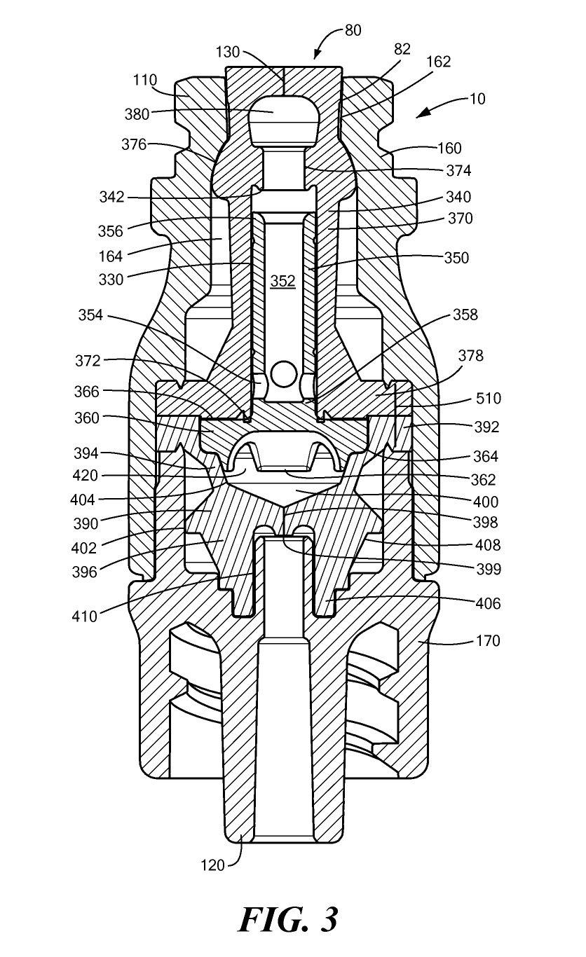

FIG. 3 schematically shows the cross section of the valve shown in FIG. 2A along the line 3-3. FIG. 3 shows the valve 10 in the closed position when no medical instrument or other instrument is inserted through the proximal port 110. As shown, the housing 100 includes an inlet housing 160 and an outlet housing 170, which connect together to form the interior of the medical valve 10. Within the interior, the medical valve 10 has a valve mechanism. The inlet housing 160 and the outlet housing 170 may be joined together in a variety of ways, including a snap-fit connection, ultrasonic welding, plastic welding, or other method conventionally used in the art.

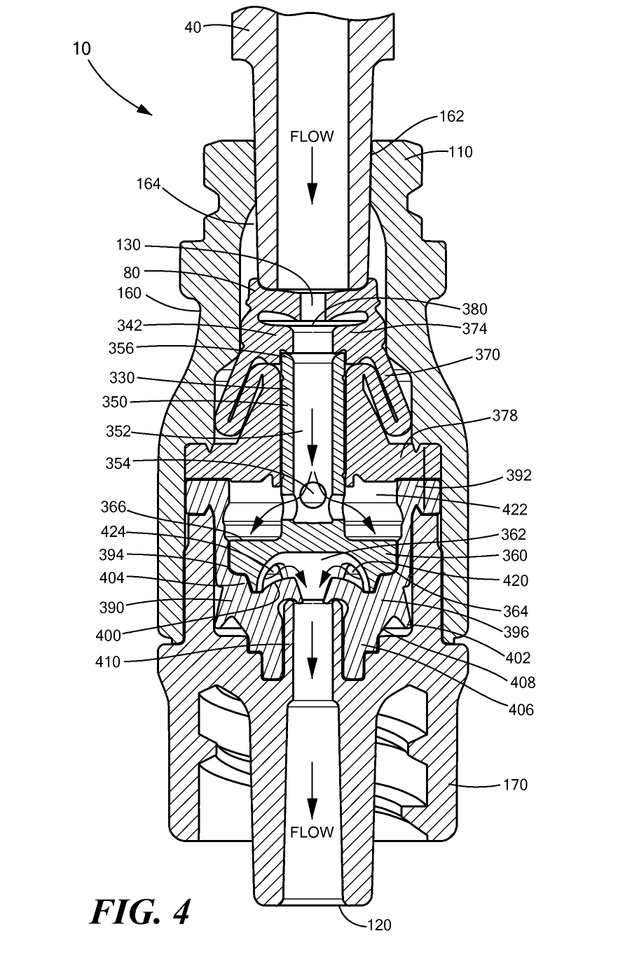

The internal valve mechanism may include a post member 330 that cooperates with a resilient member 340 to selectively open and close the valve 10. In the embodiment shown in FIG. 3, the post member 330 is typically formed from a relatively rigid material (e.g., plastic). In contrast, the resilient member 340 is typically formed from a resilient material that allows it to easily deform (e.g., silicone). Details of the interaction between the post member 330 and the resilient member 340 are discussed in greater detail below, with respect to FIG. 4.

As shown in FIG. 3, the post member 330 may include a tubular portion 350 and a head portion 360. The tubular portion 350 may be, for example, a cannula having a flow channel 352 extending through it. The tubular portion flow channel 352 may end in one or more transverse hole(s) 354 to allow fluid to enter and/or exit the flow channel 352. As discussed in greater detail below, the proximal end 356 of the tubular portion 350 may be configured to engage with a corresponding portion 342 on the resilient member 340 to help ensure proper valve actuation.

As noted above, the post member 330 may also include the post head 360, located at the distal end 358 of the tubular portion 350 (e.g., distal to the transverse holes 354). As is shown in FIG. 3, the post head 360 may have a larger outer diameter than that of the tubular portion 350 such that it extends radially outward from the tubular portion 350. As discussed in greater detail below, the post head 360 may also include one or more protrusions 362 that extend distally from a bottom surface 364 (e.g., a distal surface) of the post head 360. The protrusions 362 may interact with a portion of the resilient member 340 to open the valve 10.

The resilient member 340 may include a proximal gland 370 and a distal gland 390. As shown in FIG. 3, the proximal gland 370 may extend from the proximal port 110 to the top surface 366 (e.g., a proximal surface) of the post head 360 and circumscribe the tubular portion 350 of the post member 330. The proximal gland 370 may also form a seal against the post member 330 so as to prevent fluid from exiting or entering the transverse hole(s) 354 when the valve 10 is in the closed mode. For example, the proximal gland 370 may create a seal 372 at the top surface 366 of the post head 360. Alternatively or in addition, the proximal gland 370 may directly seal against the transverse holes 354.

The proximal gland 370 may also include the above noted proximal seal 80 at the inlet/proximal port 110 of the valve 10. As discussed above, this proximal seal 80 may include an aperture 130 that extends through its profile to provide a low-pressure seal at the valve inlet. The proximal gland 370 may also include additional features that help facilitate valve opening and closing. For example, the proximal gland 370 may include a shelf portion 374 and a rib 376. As discussed in greater detail below, the shelf portion 374 interacts with the post member 330 as the valve 10 is transitioning between the open and closed modes.

The rib 376 may be, for example, a larger diameter section of the proximal gland 370 and may function as a reinforcement and/or as a positive stop. For example, during valve 10 actuation, the rib 376 may prevent the post member 330 from extending through the shelf portion 374 and into the proximal volume 380 (e.g., the reinforcement function). Additionally, the rib 376 may help prevent the valve mechanism (e.g., the resilient member 340 and post member 330) from being urged past the closed position when the valve 10 is exposed to high back-pressures (e.g., the positive stop function).

As also shown in FIG. 3, the post member 330, at the proximal end 356 of the tubular portion 350, may be spaced from the proximal seal 80 to create a proximal volume 380 between the proximal seal 80, proximal gland 370, and the proximal surface of the post member 330. As is discussed in greater detail below and as shown in FIGS. 3 and 4, this proximal volume 380 compresses/contracts as the valve 10 transitions from the closed mode to the open mode. Conversely, the proximal volume 380 expands (e.g., back to the closed mode volume) as the valve 10 transitions from the open mode to the closed mode.

In addition to the proximal gland 370 described above and as noted above, the resilient member 340 may also include a distal gland 390 located within the outlet housing 170. The distal gland 390 has a radial flange 392 that is secured to the housing 100 (e.g., between the inlet housing 160 and the outlet housing 170) along with the radial flange 378 of the proximal gland 370. The distal gland 390 may also have a radial ledge 394 that extends from the radial flange 392 to a distal seal portion 396. When the valve 10 is in the closed mode, the post head 360 may rest on the top of the radial ledge 394.

As shown in FIG. 3, the distal seal portion 396 has a normally closed aperture 398 extending through its profile. The distal seal portion 396 has a tapered wall region 400 surrounding the normally closed aperture 398. For example, when closed, the tapered wall region 400 may be tapered distally such that the top of the distal seal portion 396 has a concave shape (e.g., as shown in FIG. 3). Alternatively, when in the closed mode, the tapered wall region 400 may be tapered proximally such that the top (e.g., proximal surface) of the distal seal portion 396 has a convex shape.

It is important to note that the tapered wall region 400 may have different configurations and/or profiles as long as the surface is generally increasing proximally or distally (e.g., as long as the top of the distal seal aperture 398 is located proximal to or distal to the inversion point 404) and permits the inversion discussed below. For example the wall may be stepped downward or stepped upward. Additionally or alternatively, the tapered wall region 400 may have an irregular profile, a frusto-conical shape, a hemispherical shape, cylindrical shape, or other undefined shape. It is also important to note that tapered wall region 400 does not have to be gradually increasing and/or decreasing or have a smooth surface. The tapered wall region 400 may have protrusions, groves, or other irregularities as long as, as a whole, the surface/wall is tapered (concave or convex, whichever the case may be).

In addition to the above, the distal gland 390 may also have additional features that aid in the transition between the open and closed modes. In some embodiments, these additional features may also help prevent back-pressure (e.g., a proximally directed pressure) from opening the distal seal aperture 398. For example, some embodiments may have one or more compression fingers 402 extending radially out from the distal gland member 390. To aid in back-pressure sealing, the compression fingers 402 may be configured such that one end of the finger 402 contacts an inner wall of the outlet housing 170. In such embodiments, the compression fingers 402 may apply a radially compressive force on the distal seal aperture 398 to pre-load the aperture 398 and increase the valve's back-pressure sealing capability. To that end, the compression fingers 402 may be slightly larger than the inner diameter of the outlet housing 170 so as to create an interference compression with the outlet housing 170.

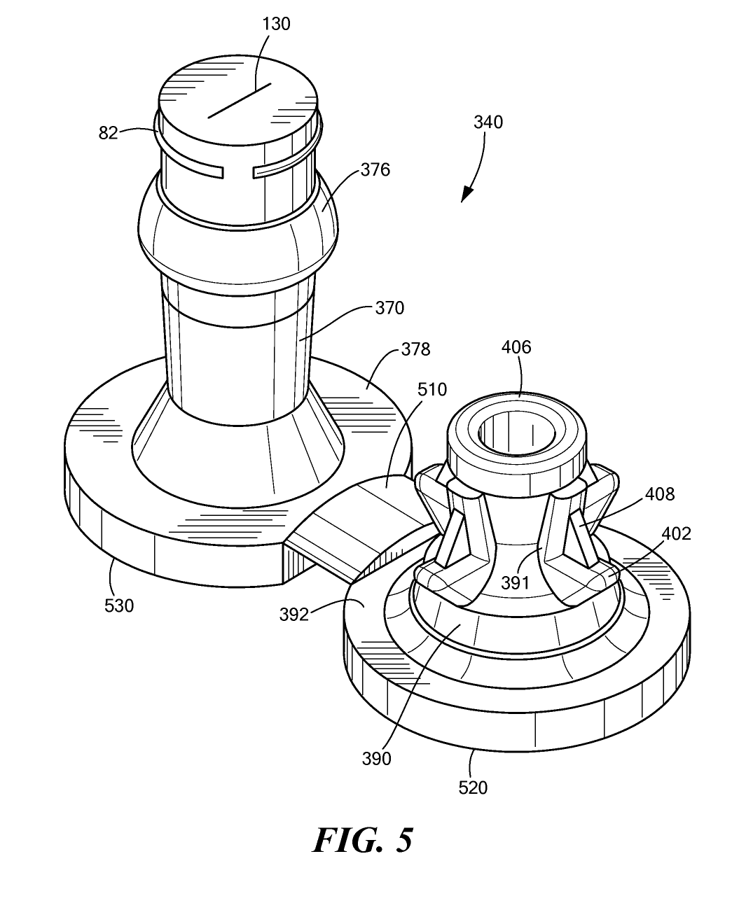

To ensure that the compression fingers 402 are able to deform, invert, and return to their at-rest/closed position (e.g., as discussed in greater detail below), the distal gland 390 may also include stiffening gussets 408. As best shown in FIG. 5, the gussets 408 may extend from the body 391 of the distal gland 390 to a point on the compression finger 402. The gussets 408 stiffen the compression fingers 402 and help the compression fingers 402 return to their at-rest position as the valve 10 closes. For example, as the compression finger(s) 402 deform distally/invert, the gussets 408 buckle. When the medical implement 40 is withdrawn, the buckling load causes the compression finger(s) 402 to spring back to their at-rest/non-inverted position to close the distal seal aperture 398. In this manner, the gussets 408 help ensure consistent performance of the valve 10.

As shown in FIGS. 3 and 4, the space between the proximal gland 370 and the distal gland 390 creates a distal volume 420 in which the post head 360 is located and into which the post member 330 moves as the valve 10 opens. As discussed in greater detail below, the distal volume 420 increases as the valve transitions from the closed mode to the open mode. In a corresponding manner, this volume 420 decreases (e.g., returns to the closed mode volume) as the valve 10 transitions from the open mode to the closed mode.

It is important to note that the post head 360 may split the distal volume 420 into two sub-volumes. The first sub-volume 422 may be located proximal to the post head 360 (e.g., between the top of the post head 360 and the bottom of the proximal gland 370) and the second sub-volume 424 may be located distal to the post head 360 (e.g., between the bottom of the post head 360 and the distal seal portion 396). When the valve 10 is in the closed mode, the first sub-volume 422 is substantially zero. However, as the post member 330 moves distally, the first sub-volume 422 increases, the second sub-volume 424 decreases, and the overall distal volume 420 increases (e.g., as the distal gland 390 deforms). Conversely, as the post member 330 moves proximally (e.g., during valve closing), the first sub-volume 422 decreases (e.g., back towards the substantially zero volume), the second sub-volume 424 increases, and the overall distal volume 420 decreases (e.g., as the distal gland 390 returns to the at-rest/closed position).

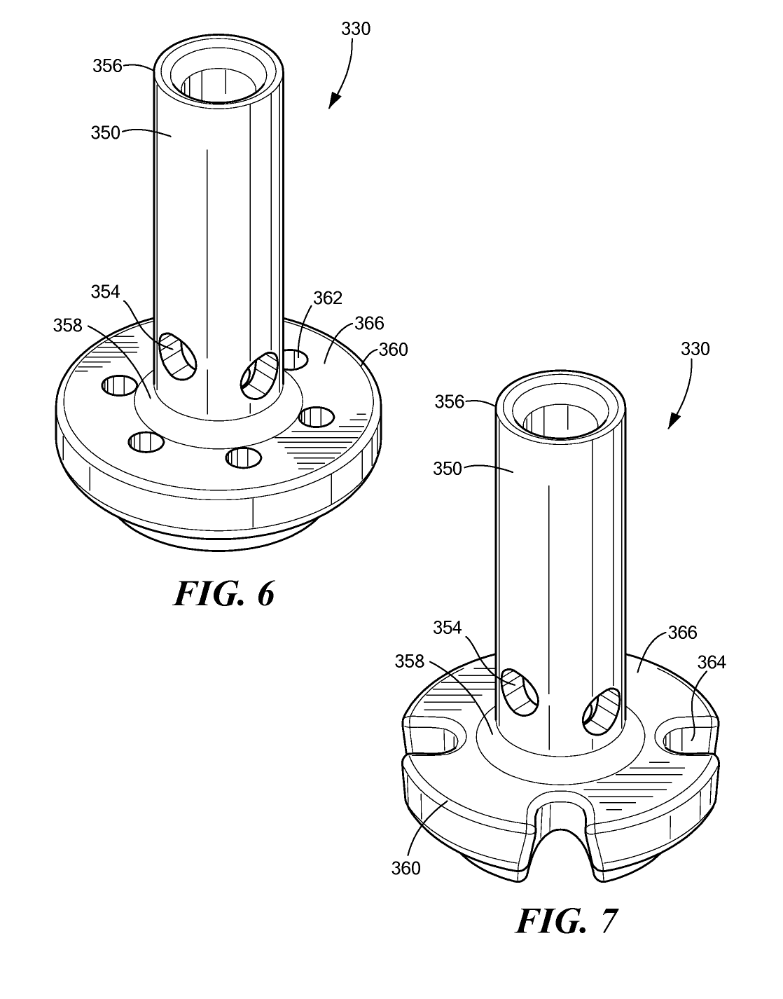

In order to allow fluid to pass back and forth between the first sub-volume 422 and the second sub-volume 424 (e.g., to allow for sub-volume expansion and contraction and to allow fluid to be transferred to/from the patient 30), the post head 360 is configured to allow fluid to pass through it. For example, the post head 360 may have holes 362 passing through it (e.g., as shown in FIG. 6), or grooves 364 cut into the edge of the post head 360 (e.g., as shown in FIG. 7). It should be noted that the transfer of fluid from one side of the post head 360 to the other prevents a vacuum from developing as the post member 330 moves proximally within the housing.

As mentioned above and illustrated in FIG. 4, distal movement of the post member 330 opens the valve 10. In particular, when a medical practitioner or other user inserts a medical instrument 40 into the valve 10, the proximal gland 370 begins to deform and move distally within the proximal housing 160. The proximal gland's deformation and distal movement, in turn, causes the proximal volume 380 to contract. It is important to note that the proximal seal aperture 130 is expected to remain closed until the proximal seal 80 exits the luer taper region 162 of the inlet housing 160 and enters the expansion region 164. As the proximal seal 80 enters the expansion region 164, the proximal seal aperture 130 will open.

Upon further distal movement of the medical instrument 40 into the valve 10, the bottom/distal portion of the shelf 374 (e.g., portion 342) will make contact with the post member 330 and begin to move the post member 330 distally within the housing 100. As mentioned above, the proximal end 356 of the tubular portion 350 may be configured to engage with the shelf 374. To that end (as shown in FIGS. 3 and 4), the proximal end 356 of the post member 330 may be angled and/or chamfered such that it corresponds with and engages with the underside (e.g., portion 342) of the shelf 374. As the post member 330 moves distally within the housing, the transverse hole(s) 354 will be exposed to the distal volume 420 (e.g., the first sub-volume 422, FIG. 4).

Additionally, as the post member 330 moves distally, the post head protrusions 362 will begin to deform the distal gland 390. For example, as shown in FIG. 4, the ledge 394 deforms radially outward and the tapered wall region 400 deforms distally at inversion point 404. The distal deformation of the tapered wall region 400 at inversion point 404 causes the area of the tapered wall region radially inward of the inversion point 404 to essentially invert and deform proximally (e.g., to form the convex area shown in FIG. 4).

As also shown in FIG. 4, as the deformation of the distal gland 390 continues, the compression fingers 402 will be deformed and angled distally, causing the distal gland aperture 398 to open. Additionally, it should be noted that the deformation of the distal gland 390 essentially inverts various portions the distal gland 390. For example, the tapered wall region 400 which, as mentioned above, may form a concave area around the distal gland aperture 398 inverts (e.g., at inversion point 404) from the concave shape to a generally convex shape. Additionally, as noted above, the compression fingers 402 invert and angle distally. When the compression fingers 402 are in the inverted position, the compression fingers 402 do not apply a radially compressive force on the distal seal aperture 398 sufficient to keep the distal seal aperture 398 closed.

It should be noted that, in this context, the term “invert” or “inversion” refers to when components change position relative to other components. For example, the inversion of the tapered wall region 400 causes a relative change in position of the distal seal aperture 398 with respect to the inversion point 404. In particular, when in the non-inverted state, the top of the distal seal aperture 398 is distal to the inversion point 404. However, as the tapered wall region 400 inverts, the inversion point 404 moves distally such that, when in the inverted state, the top of the distal seal aperture 398 is proximal to the inversion point 404 (see FIGS. 3 and 4).

As mentioned above, the distal gland aperture 398 opens as the distal gland 390 deforms and the compression fingers 402 invert/deform downward. To aid in distal gland aperture 398 opening and distal gland 390 inversion, the outlet housing 170 may include an outlet protrusion 410, around the outlet, that extends proximally into the outlet housing 170. In such embodiments, the distal gland 390 may have a distally extending portion 406 that circumscribes the outlet protrusion 410. Therefore, as the valve 10 transitions from the closed mode to the open mode, the post member 330 deforms the distal gland 390 over the protrusion 410, which, in turn, aids in distal gland aperture 398 opening. For example, as the post member 330 applies the distally directed force on the tapered wall region 400 (e.g., radially outward from the outlet protrusion 410), the outlet protrusion 410 may act as a stop and/or a anchoring point about which the tapered wall region 400 may deform (e.g., to open the distal gland aperture 398).

Once the valve 10 is in the open mode (e.g., after the distal seal aperture 398 is open), the medical practitioner or other user may transfer fluid to and/or from the patient. When fluid is transferred to and/or from the patient 30, the fluid passes through a fluid path within the valve 10. As the name suggests, the fluid path is the path the fluid takes as it passes through the valve 10. As shown in FIG. 4 and denoted by the flow arrows, the fluid path includes the proximal aperture 130, the proximal volume 380, the tube portion fluid channel 352, the distal volume 420, and the distal seal aperture 398.

Upon disconnection and withdrawal of the medical implement 40, the resilient characteristics of the proximal gland 370 and the distal gland 390 urge the valve 10 from the open mode shown in FIG. 4 back to the closed mode shown in FIG. 3. In particular, as the proximal gland 370 and the distal gland 390 begin to return their at-rest states, their resiliency causes the post member 330 to begin moving proximally within the valve 10. As the post member 330 moves proximally, the tapered wall region 400 and the compression fingers 402 return to their closed/at rest position, causing the distal gland aperture 398 to close.

It is important to note that the configuration of the distal gland 390 and the manner in which it deforms helps the distal gland aperture 398 close very early in the return stroke of the medical implement 40. Specifically, minimal proximal movement of the post member 330 causes the tapered wall region 400 and the compression fingers 402 to return to their non-inverted states. This early inversion causes the distal gland aperture 398 to close. The amount of longitudinal movement of the medical implement 40 required to close the distal gland aperture 398, thus, preferably is much less than that required to open the distal gland aperture 398.

For example, in some embodiments, the total stroke distance of the medical implement 40 (e.g., as it is being inserted and/or withdrawn) may be approximately 0.25 inches. As the valve 10 transitions from the closed mode to the open mode, the distal seal aperture 398 may not open until the medical implement 40 has been inserted 0.20 inches or 80% of the total stroke distance. Conversely, as the valve transitions from the open mode to the closed mode, the distal seal aperture 398 may close within the first 0.05 inches of travel (or the within the first 20% of the total stroke distance). In other words, the travel distance required to close the distal seal aperture 398 may be only 25% of the distance required to open the distal seal aperture 398 (e.g., 0.05 inches is approximately 25% of 0.20 inches).

It is important to note that the above distances and percentages are merely examples and the total stroke distance, the distance required to open the distal seal aperture 398, and the distance required to close the distal seal aperture 398 may be higher or lower. For example, the total stroke distance may be greater or less than 0.25 inches (e.g., it may range from 0.22 inches to 0.27 inches). Additionally or alternatively, the distance required to open the distal seal aperture 398 may be greater than or less than the 0.2 inches (80% of the total travel distance) mentioned above. Similarly, the distance required to close the distal seal aperture 398 may be greater than or less than the 0.05 inches (20% of the total travel distance) mentioned above. For example, the distance required to open the aperture 398 may range from 60% to 90% of the total stroke distance and the distance required to close the aperture 398 may be 10% to 40% of the total stroke distance. The range to close the distal seal aperture 398 may also be 20% to 30%, 10% to 30%, 10% to 20%, 5% to 10% or less than 10% of the total stroke distance.

The “quick-close” nature of the distal gland aperture 398 helps reduce the amount of drawback upon disconnection. In particular, once the distal gland aperture 398 is closed, further proximal movement of the post member 330 or changes in the pressure and/or fluid volume proximal to the distal gland aperture 398 should not impact fluid movement/flow at the outlet. In other words, even if the volume increases or a pressure builds up proximal to the distal seal aperture 398 after it closes, the displacement at the outlet will not be impacted.

Although FIG. 4 shows a distal gland 390 having four compression fingers 402, other embodiments may utilize a different number of fingers 402. For example, some embodiments may only utilize two compression fingers 402, while others may use three or more. It is also important to note that the number and location of the compression fingers 402 may be dependent upon the configuration of the distal gland aperture 398. For example, if the distal gland aperture 398 is a slit, the distal gland 390 may have two compression fingers 402 (one located on either side of the slit). Alternatively, if the distal gland aperture 398 is a three axis trocar type slit, the distal gland 390 may have three compression fingers 402 located 120 degrees apart and positioned between the slit axes.

As mentioned above, some embodiments of the present invention may exhibit a neutral fluid displacement upon connection and/or disconnection of the medical implement 40 (e.g., upon opening and closing of the valve 10). In addition to the quick-close nature of the distal gland aperture 398, the multiple variable volume regions (e.g., proximal volume 380 and distal volume 420) discussed above may also help achieve neutral fluid displacement. For example, as discussed above, the proximal volume 380 decreases and the distal volume 420 increases (e.g., as the distal gland 390 expands radially outward and downward as it deforms) as the valve 10 transitions from the closed mode to the open mode. Conversely, the proximal volume 380 increases and the distal volume 420 decreases as the valve 10 transitions from the open mode to the closed mode.

To that end, the fluid contained within the valve 10 may move toward and/or between the proximal volume 380 and distal volume 420 as one volume expands and the other contracts. For example, as the valve 10 is transitioning from the open mode to the closed mode, some of the fluid within the contracting distal volume 420 may flow toward the transverse hole(s) 354, through the post member fluid path 352, and into the proximal volume 380 as the proximal volume 380 expands. Similarly, as the valve 10 opens, the fluid contained within the proximal volume 380 may be expelled from the proximal volume 380 as it contracts. The expelled fluid may then flow into and/or toward the distal volume 420 as it expands (e.g., by entering the post member fluid path 352 and exiting through the transverse hole(s) 354).

In some embodiments, the changes in volume of both the proximal volume 380 and the distal volume 420 may be substantially equal. In other words, as the valve 10 opens, the distal volume 420 will increase by substantially the same amount that the proximal volume 380 decreases. Similarly, when the valve 10 closes, the proximal volume will increase by substantially the same amount that the distal volume 420 decreases. In such embodiments, the total fluid volume within the valve 10 will remain substantially constant as the valve transitions between the open and closed modes, thereby creating a substantially neutral fluid displacement at the outlet during both opening and closing of the valve 10.

In other embodiments, the proximal volume 380 and the distal volume 420 offset one another primarily when the valve 10 is in the open mode and up until the time that the distal seal aperture 398 closes. In such embodiments, once the distal seal aperture 398 closes (as noted above), any volume changes will not impact the fluid displacement at the outlet 120. Therefore, if the proximal volume 380 expands more or at a faster rate than the distal volume 420 contracts (e.g., increasing the total volume), there will be no drawback into the outlet 120. Similarly, if the proximal volume expands less or slower than the distal volume 420 contracts, there will be no positive displacement at the outlet 120.

It is important to note that, although the resilient member 340 is described above as having two pieces (e.g., the proximal gland 370 and the distal gland 390), the resilient member 340 may be manufactured as a single piece. For example, as shown in FIG. 5, the resilient member 340 may be manufactured with an integrally molded tab or hinge 510 between the proximal gland 370 and the distal gland 390. During assembly, the distal gland 390 may be folded about the hinge 510 such that a proximal face 520 of the distal gland 390 abuts a distal face 530 of the proximal gland 370 (e.g., as shown in FIGS. 3 and 4).

In some embodiments, the proximal gland 370 and the distal gland 390 may become cross-linked. For example, during gamma sterilization, the proximal face 520 of the distal gland 390 and the distal face 530 of the proximal gland 370 may become cross-linked such that the two component essentially form a single piece. Additionally or alternatively, the components may be joined together in other ways including, but not limited to, using adhesives or plasma discharge treatments.

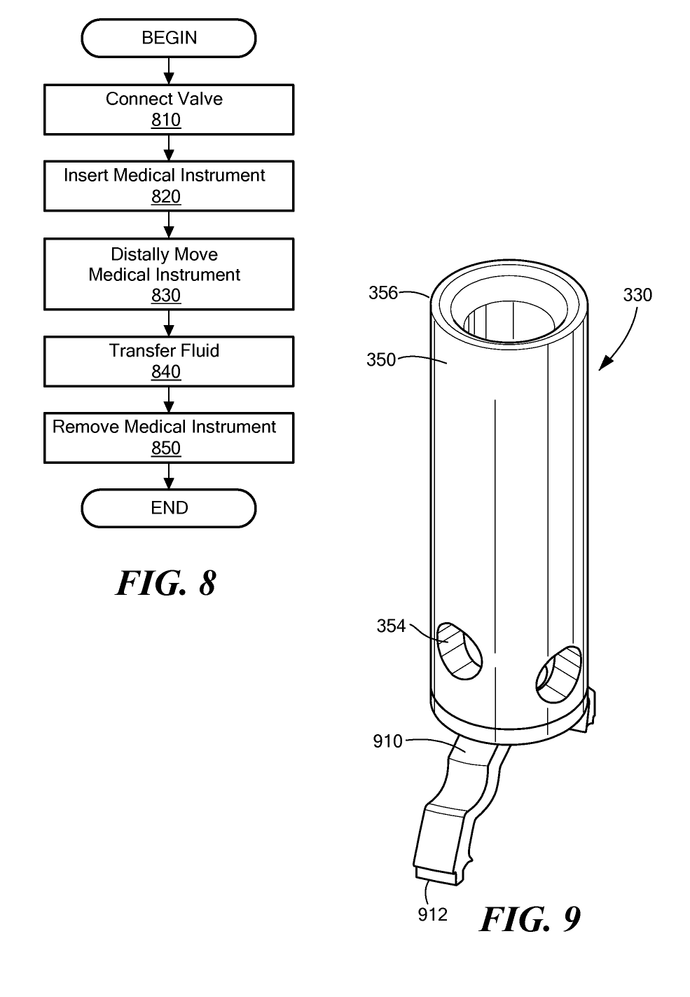

FIG. 8 shows a process illustrating one of a plurality of illustrative uses of the medical valve 10. It is important to reiterate that, according to good medical practice, the proximal port 110 and distal port 120 of medical valve 10 should be cleaned (e.g., swabbed) prior to any connection and after any disconnection. After properly swabbing the distal port 120 of the medical valve 10 (i.e., the gland is generally flush with or extends above the inlet), a medical practitioner 20 connects the medical valve 10 to the patient 30 (step 810). To do so, the medical practitioner 20 may connect the distal port 120 of the medical valve 10 to the catheter 70, which terminates at a needle inserted into the patient 30 (see FIG. 1).

After connecting the valve 10 to the patient 30, the medical practitioner 20 swabs the valve proximal port 110 and inserts the medical instrument 40 into the proximal port 110 (step 820). As the medical practitioner 20 moves the medical instrument distally (step 830) into the medical valve 10, the instrument 40 will begin to deform the proximal seal 80 and move it distally to open the proximal aperture 130, as discussed above. As the proximal seal 80 deforms, the proximal volume 380 will collapse/contract and the shelf portion 374 will contact the post member 330 and begin to move the post member 330 distally to unseal the transverse hole(s) 354. Further insertion of the instrument continues to move the post member 330 distally and deforms/inverts the distal gland 390 and opens the distal seal aperture 398, as discussed above. When the distal seal aperture 398 opens, there is fluid communication between the proximal port 110 and the distal port 120. At this point, the valve 10 is open. The instrument may, in some instances, be inserted further even after the aperture 398 opens. In that case, the valve 10, should still function as described.

After opening the valve 10, the medical practitioner 20 can transfer fluids to or from the patient (step 840). For example, if the medical practitioner 20 wishes to administer a medication to the patient 30, he/she may depress the medical instrument plunger 40 (e.g., for a syringe) and transfer the medication into the patient 30. Alternatively, the medical practitioner 20 may withdraw blood from the patient 30.

After completing the fluid transfer(s), the medical practitioner 20 can remove the medical instrument (step 850). As discussed above, the medical practitioner 20 should take care not to squeeze the sides of the medical instrument 40. Doing so may create a false positive or negative displacement at the distal port 120 of the medical valve 10. If done properly, removal of the medical instrument 40 may result in a substantially neutral or a positive displacement at the valve distal port 120.

As discussed above with reference to FIGS. 3 and 4, the post member 330 will begin to move proximally as the medical practitioner 30 withdraws the medical instrument 40 from the medical valve 10 (e.g., as the proximal gland 370 and distal gland 390 begin to return to their at rest states). As the post member 330 moves proximally towards its at rest position (e.g., closed position), the tapered wall region 400 and the compression fingers 402 will return to their non-inverted states to quickly close the distal gland aperture 398 and fluidly disconnect the proximal port 110 and distal port.

The fluid path through the valve 10 has a closed mode volume when the valve is in the closed mode and an open mode volume. The open mode volume may be the volume at any point after the distal seal aperture 398 opens. For example, the open mode volume may be the volume of the fluid path just after the distal seal aperture 398 opens. Alternatively, the open mode volume may be the volume at which the medical implement 40 can no longer be inserted into the valve 10 (e.g., when the medical implement is fully inserted). The open mode volume may also be the volume at any point between immediately after the distal seal aperture 398 opens and maximum insertion of the medical implement 40.

It is important to note that, even after the distal seal aperture 398 is open, further insertion of the medical implement 40 may continue to move the post member 330. This additional distal movement of the post member 330 may further deform the proximal gland 370 and the distal gland 390 which, in turn, may also change the volumes (e.g., proximal volume 380 and distal volume 420) within the valve 10. However, for the purposes of achieving the neutral displacement discussed above, the open mode volume of the fluid path need only be substantially equal to the closed mode volume at a single point after the distal seal aperture 398 opens (e.g., immediately after opening or when the medical implement 40 is fully inserted.

Thus, in various embodiments, between the open state and the quick-closed state (e.g., the state at which the distal seal aperture 398 first closes), the volume within the fluid path remains substantially constant to produce a neutral drawback. In other embodiments, the volume within the fluid path may increase to create a negative displacement (e.g., s drawback) or decrease to create a positive displacement at the outlet 120.

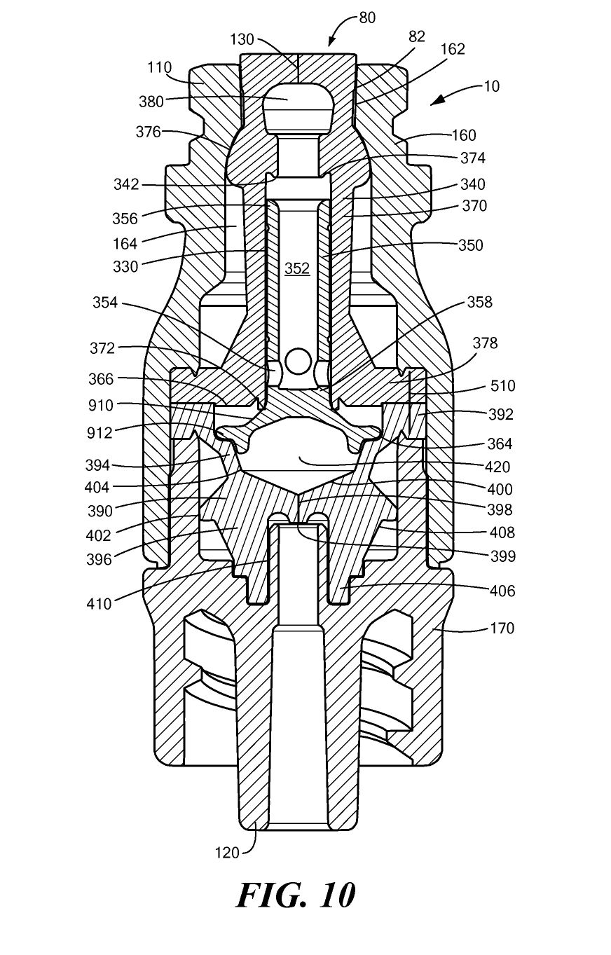

It should be noted that, although the above described embodiments contain a post member 330 with a post head 360, other embodiments may utilize different post member 330 configurations. For example, as shown in FIG. 9, instead of the post head 360 and protrusions 362, the post member 330 may have one or more legs 910 extending distally from the distal end of the post member 330.

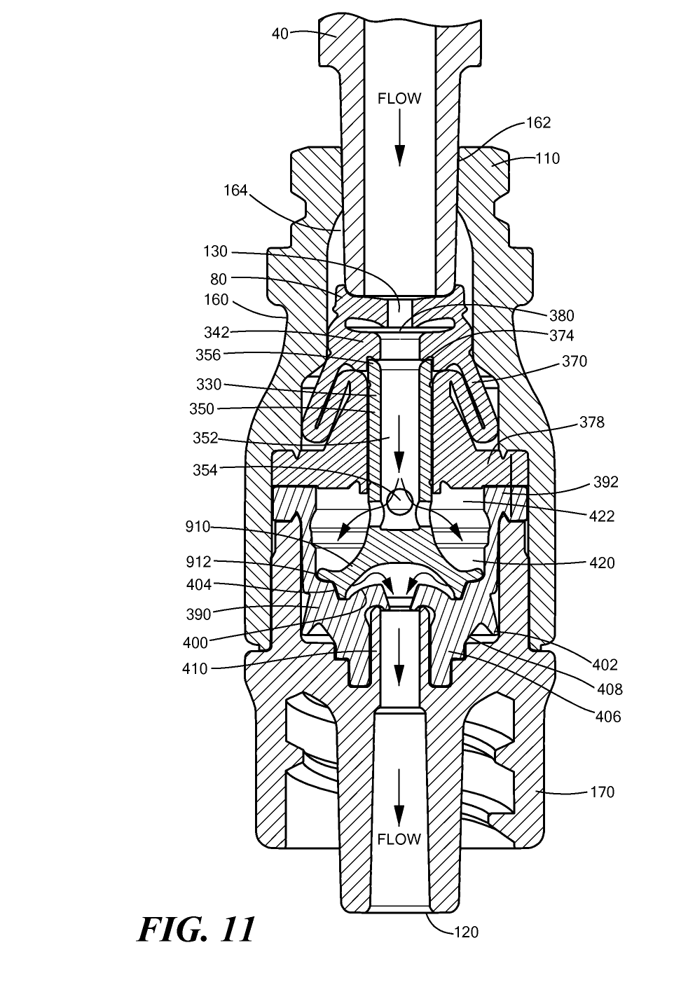

As shown in FIGS. 10 and 11, the legs 910 may be angled such that the ends 912 of the legs 910 contact the distal gland 390, for example, at the ledge 394. In operation, the legs 910 act in a similar manner as the post head protrusions 362 discussed above. In particular, as the post member 330 moves distally, the legs 910 will apply a pressure on the distal gland 390 to deform/invert the tapered wall region 400 and compression fingers 402. This, in turn, will open the distal gland aperture 398.

It is important to note that, although the legs 910 are described above as contacting the ledge 394, the legs 910 (and/or the post head protrusions 362) may contact other areas of the distal gland 390. For example, in some embodiments, the legs 910 may be angled such that they contact the tapered wall region 400 (e.g., as opposed to the ledge 394).

Various embodiments of the present invention may also include features that help keep the distal seal aperture 398 closed in the presence of a back-pressure or proximally directed force/pressure. For example, the distal seal 390 may include a thickened portion or a protrusion 399 extending distally into the outlet (see FIG. 3). In such embodiments, the proximally directed pressure will apply a proximally directed force on the distal surface of the distal seal 390 and a radially compressive force on the protrusion 399. The radially compressive force helps to keep the distal seal aperture 398 closed. Therefore, the greater the proximally directed pressure, the greater the radially compressive force applied to the protrusion 399. In this manner, the protrusion 399 acts to provide the valve 10 with a dynamic back pressure seal.

Although the above discussion discloses various exemplary embodiments of the invention, it should be apparent that those skilled in the art can make various modifications that will achieve some of the advantages of the invention without departing from the true scope of the invention.

What is claimed is:

1. A medical valve having an open mode that permits fluid flow, and a closed mode that prevents fluid flow, the medical valve comprising:
a housing having an inlet and an outlet;
a valve member within the housing and having a distal seal member with a normally closed aperture therethrough, insertion of a medical implement into the inlet opening the aperture and fluidly connecting the inlet and outlet via a fluid path between the inlet and the outlet, withdrawal of the medical implement closing the aperture and fluidly disconnecting the inlet and outlet;
a first variable volume region; and
a second variable volume region longitudinally spaced from the first variable volume region, the first and second variable volume regions being part of the fluid path, the first variable volume region contracting upon withdrawal of the medical implement, the second variable volume region expanding upon withdrawal of the medical implement,
the fluid path having a closed volume before insertion of the medical implement and an open volume when in the open mode, the closed volume being substantially equal to the open volume.

2. A medical valve according to claim 1, wherein the volumes of the first and second variable volume regions are configured to respectively contract and expand to produce a substantially neutral fluid displacement at the outlet during disconnection of the medical implement.

3. A medical valve according to claim 1, wherein the fluid path open volume is the volume when the medical implement is inserted to its farthest point.

4. A medical valve according to claim 1, wherein the fluid path open volume is the volume when the medical implement is only partially inserted.

5. A medical valve according to claim 1, wherein the distal seal member bounds the second variable volume region when in the closed mode.

6. A medical valve according to claim 1, wherein the fluid path open volume is the volume when the aperture first opens.

7. A medical valve according to claim 1, wherein the fluid path open volume is the volume when the aperture is fully open.

8. A medical valve according to claim 1, wherein the valve member includes:
a post member moveably mounted within the housing, the post member being distally moveable within the housing to fluidly connect the inlet and outlet after insertion of the medical implement into the inlet, the post member being proximally moveable within the housing to fluidly disconnect the inlet and outlet upon withdrawal of the medical implement.

9. A medical valve according to claim according to claim 8, wherein the post member has a flow channel therethrough, the flow channel fluidly communicating the first volume and the second volume when in the open mode, thereby allowing fluid from the first volume to enter the second volume as the first volume contracts.

10. A medical valve according to claim 8, wherein the post member comprises a tube portion and a head portion, the head portion protruding radially outward from the tube portion.

11. A medical valve according to claim 10, wherein the head portion has a proximally facing surface and a distally facing surface and a post head channel extending between the proximally facing surface and the distally facing surface.

12. A medical valve according to claim 11, wherein the first variable volume has a first sub-volume and a second sub-volume, the first and second sub-volume being separated by the head portion, the first sub-volume being proximal to the proximally facing surface, the second sub-volume being distal to the distally facing surface, the first sub-volume contracting upon withdrawal of the medical implement, the second sub-volume expanding upon withdrawal of the medical implement, the post head channel fluidly connecting the first and second sub-volumes.

13. A medical valve according to claim 10, wherein the head portion has a plurality of distally extending protrusions, the protrusions applying a force on the distal seal to open the aperture after the medical implement is inserted.

14. A medical valve according to claim 8, wherein the post member comprises a tube portion and a plurality of legs extending distally from a distal end of the tube portion, the leg portions applying a force on the distal seal to open the aperture after medical implement is inserted into the inlet.

15. A medical valve according to claim 14, wherein the plurality of legs apply a radially directed force and a longitudinally directed force on the distal seal as the post member moves distally, the radially directed force and longitudinally directed force opening the aperture.

16. A medical valve according to claim 1, wherein at least a portion of the distal seal member inverts from a first shape to a second shape as the aperture opens.

17. A medical valve according to claim 1, wherein the valve further includes a proximal seal member with an normally closed aperture therethrough, insertion of the medical implement moving the proximal seal member distally and opening the proximal seal aperture.

18. A medical valve according to claim 17, wherein the proximal seal member is swabbable when the valve is in the closed mode.

19. A medical valve according to claim 17, wherein the first variable volume is located between the post member and the distal seal, the second variable volume being located between the post member and the proximal seal.

20. A medical valve according to claim 1, wherein the distal seal includes a protrusion extending distally from the distal seal, the normally closed aperture passing through the protrusion, the protrusion providing a dynamic back pressure seal.

21. A medical valve according to claim 1, wherein the first variable volume expands after insertion of the medical implement and the second variable volume contracts after insertion of the medical implement.

22. A medical valve according to claim 1, wherein a substantially neutral fluid displacement occurs at the outlet during connection of the medical implement.

23. A medical valve according to claim 1, wherein the medical implement travels a distal stroke distance to open the aperture and a proximal stroke distance to close the aperture, the distal stroke distance being the distance from initial connection of the medical implement to the point at which the aperture first opens, the proximal stroke distance being the distance from the point at which the medical implement is fully inserted to the point at which the aperture first closes, the proximal stroke distance being less then the distal stroke distance.

24. A medical valve according to claim 23, wherein the proximal stroke distance is 25% of the distal stroke distance.

25. A method comprising:
connecting a medical valve to a patient, the medical valve comprising a housing having an inlet and an outlet, and a valve member having a distal seal member with a normally closed aperture therethrough, the medical valve also having a first variable volume region and a second variable volume region longitudinally spaced from the first variable volume region;
inserting a medical implement through the inlet;
moving the medical implement distally within the housing to transition the valve from an open mode to a closed mode by opening the normally closed aperture and fluidly connecting the inlet and outlet via a fluid path between the inlet and the outlet, the first and second variable volume regions being part of the fluid path;
transferring fluid between the medical implement and the patient through the valve; and
moving the medical implement proximally within the housing to fluidly disconnect the inlet and outlet by closing the aperture, the first variable volume region contracting upon withdrawal of the medical implement, the second variable volume region expanding upon withdrawal of the medical implement,
the fluid path having a closed volume before insertion of the medical implement and an open volume when in an open mode, the closed volume being substantially equal to the open volume.

26. A medical valve according to claim 25, wherein the volumes of the first and second variable volume regions are configured to respectively contract and expand to produce a substantially neutral fluid displacement at the outlet during disconnection of the medical implement.

27. A medical valve according to claim 25, wherein the fluid path open volume is the volume when the medical implement is inserted to its farthest point.

28. A medical valve according to claim 25, wherein the fluid path open volume is the volume when the medical implement is only partially inserted.

29. A medical valve according to claim 25, wherein the distal seal member bounds the second variable volume region when in the closed mode.

30. A medical valve according to claim 25, wherein the fluid path open volume is the volume when the aperture first opens.

31. A medical valve according to claim 25, wherein the fluid path open volume is the volume when the aperture is fully open.

32. A method according to claim 25, wherein the valve member includes:
a post member moveably mounted within the housing, the post member being moveable distally within the housing to fluidly connect the inlet and outlet after insertion of the medical implement into the inlet, the post member being moveable proximally within the housing to fluidly disconnect the inlet and outlet upon withdrawal of the medical implement.

33. A method according to claim according to claim 32, wherein the post member has a flow channel therethrough, the flow channel fluidly communicating the first volume and the second volume when in the open mode, thereby allowing fluid from the first volume to enter the second volume as the first volume contracts.

34. A method according to claim 32, wherein the post member comprises a tube portion and a head portion, the head portion protruding radially outward from the tube portion.

35. A method according to claim 34, wherein the head portion has a proximally facing surface and a distally facing surface and a post head channel extending between the proximally facing surface and the distally facing surface.

36. method according to claim 35, wherein the first variable volume has a first sub-volume and a second sub-volume, the first and second sub-volume being separated by the head portion, the first sub-volume being proximal to the proximally facing surface, the second sub-volume being distal to the distally facing surface, the first sub-volume contracting upon withdrawal of the medical implement, the second sub-volume expanding upon withdrawal of the medical implement, the post head channel fluidly connecting the first and second sub-volumes.

37. A method according to claim 34, wherein the head portion has a plurality of protrusions extending distally therefrom, the protrusions applying a force on the distal seal to open the aperture after the medical implement is inserted.

38. A method according to claim 32, wherein the post member comprises a tube portion and a plurality of legs extending distally from a distal end of the tube portion, the leg portions applying a force on the distal seal to open the aperture after medical implement is inserted into the inlet.

39. A method according to claim 38, wherein the plurality of legs apply a radially directed force and a longitudinally directed force on the distal seal as the post member moves distally, the radially directed force and longitudinally directed force opening the aperture.

40. A method according to claim 25, wherein at least a portion of the distal seal member inverts from a first shape to a second shape as the aperture opens.

41. A method according to claim 25, wherein the first variable volume expands after insertion of the medical implement and the second variable volume contracts after insertion of the medical implement.

42. A medical valve according to claim 25, wherein a substantially neutral fluid displacement occurs at the outlet during connection of the medical implement.

43. A medical valve having an open mode that permits fluid flow, and a closed mode that prevents fluid flow, the medical valve comprising:
a housing having an inlet and an outlet;
a valve means within the housing and having a distal sealing means with a normally closed aperture therethrough, insertion of a medical implement into the inlet opening the aperture and fluidly connecting the inlet and outlet via a fluid path between the inlet and the outlet, withdrawal of the medical implement closing the aperture and fluidly disconnecting the inlet and outlet;
a first means for forming a first variable volume region; and
a second means for forming a second variable volume region longitudinally spaced from the first variable volume region, the first and second variable volume regions being part of the fluid path, the first variable volume region contracting upon withdrawal of the medical implement, the second variable volume region expanding upon withdrawal of the medical implement,
the fluid path having a closed volume before insertion of the medical implement and an open volume when in an open mode, the closed volume being substantially equal to the open mode.

44. A medical valve according to claim 43, wherein the volumes of the first and second variable volume regions are configured to respectively contract and expand to produce a substantially neutral fluid displacement at the outlet during disconnection of the medical implement.

45. A medical valve according to claim 43, wherein the fluid path open volume is the volume when the medical implement is inserted to its farthest point.

46. A medical valve according to claim 43, wherein the fluid path open volume is the volume when the medical implement is only partially inserted.

47. A medical valve according to claim 43, wherein the distal seal member bounds the second variable volume region when in the closed mode.

48. A medical valve according to claim 43, wherein the fluid path open volume is the volume when the aperture first opens.

49. A medical valve according to claim 43, wherein the fluid path open volume is the volume when the aperture is fully open.

50. A medical valve according to claim 43, wherein the valve means includes:
a post member moveably mounted within the housing, the post member being moveable distally within the housing to fluidly connect the inlet and outlet after insertion of the medical implement into the inlet, the post member being moveable proximally within the housing to fluidly disconnect the inlet and outlet upon withdrawal of the medical implement.

51. A medical valve according to claim according to claim 50, wherein the post member has a flow channel therethrough, the flow channel fluidly communicating the first variable volume and the second variable volume when in the open mode, thereby allowing fluid from the first volume to enter the second volume as the first volume contracts.

52. A medical valve according to claim 50, wherein the post member comprises a tube portion and a head portion, the head portion protruding radially outward from the tube portion.

53. A medical valve according to claim 52, wherein the head portion has a proximally facing surface and a distally facing surface and a post head channel extending between the proximally facing surface and the distally facing surface.

54. A medical valve according to claim 52, wherein the first variable volume has a first sub-volume and a second sub-volume, the first and second sub-volume being separated by the head portion, the first sub-volume being proximal to the proximally facing surface, the second sub-volume being distal to the distally facing surface, the first sub-volume contracting upon withdrawal of the medical implement, the second sub-volume expanding upon withdrawal of the medical implement, the post head channel fluidly connecting the first and second sub-volumes.

55. A medical valve according to claim 52, wherein the head portion has a plurality of protrusions extending distally therefrom, the protrusions applying a force on the distal seal to open the aperture after the medical implement is inserted.

56. A medical valve according to claim 50, wherein the post member comprises a tube portion and a plurality of legs extending distally from a distal end of the post member, the leg portions applying a force on the distal seal to open the aperture after medical implement is inserted into the inlet.

57. A medical valve according to claim 56, wherein the plurality of legs apply a radially directed force and a longitudinally directed force on the distal sealing means as the post member moves distally, the radially directed force and longitudinally directed force opening the aperture.

58. A medical valve according to claim 43, wherein at least a portion of the distal sealing means inverts from a first shape to a second shape as the aperture opens.

59. A medical valve according to claim 43, wherein the first variable volume expands upon insertion of the medical implement and the second variable volume contracts upon insertion of the medical implement.

60. A medical valve according to claim 43, wherein a substantially neutral fluid displacement occurs at the outlet during connection of the medical implement.

61. A medical valve having an open mode that permits fluid flow, and a closed mode that prevents fluid flow, the medical valve comprising:
a housing having an inlet and an outlet;
a valve member within the housing and having a distal seal member with a normally closed aperture therethrough, insertion of a medical implement into the inlet opening the aperture and fluidly connecting the inlet and outlet via a fluid path between the inlet and the outlet, withdrawal of the medical implement closing the aperture and fluidly disconnecting the inlet and outlet;
a first variable volume region; and
a second variable volume region longitudinally spaced from the first variable volume region, the first and second variable volume regions being part of the fluid path, the first variable volume region contracting upon withdrawal of the medical implement, the second variable volume region expanding upon withdrawal of the medical implement,
the fluid path having a closed volume before insertion of the medical implement and an open volume when in the open mode, the closed volume being less than the open volume.

62. A medical valve according to claim 61, wherein the volumes of the first and second variable volume regions are configured to respectively contract and expand to produce a positive fluid displacement at the outlet during disconnection of the medical implement.

63. A medical valve according to claim 61, wherein the valve member includes:
a post member moveably mounted within the housing, the post member being distally moveable within the housing to fluidly connect the inlet and outlet after insertion of the medical implement into the inlet, the post member being proximally moveable within the housing to fluidly disconnect the inlet and outlet upon withdrawal of the medical implement.

64. A medical valve according to claim according to claim 63, wherein the post member has a flow channel therethrough, the flow channel fluidly communicating the first volume and the second volume when in the open mode, thereby allowing fluid from the first volume to enter the second volume as the first volume contracts.

65. A medical valve according to claim 63, wherein the post member comprises a tube portion and a head portion, the head portion protruding radially outward from the tube portion.

66. A medical valve according to claim 65, wherein the head portion has a proximally facing surface and a distally facing surface and a post head channel extending between the proximally facing surface and the distally facing surface.

67. A medical valve according to claim 66, wherein the first variable volume has a first sub-volume and a second sub-volume, the first and second sub-volume being separated by the head portion, the first sub-volume being proximal to the proximally facing surface, the second sub-volume being distal to the distally facing surface, the first sub-volume contracting upon withdrawal of the medical implement, the second sub-volume expanding upon withdrawal of the medical implement, the post head channel fluidly connecting the first and second sub-volumes.

68. A medical valve according to claim 65, wherein the head portion has a plurality of distally extending protrusions, the protrusions applying a force on the distal seal to open the aperture after the medical implement is inserted.

69. A medical valve according to claim 63, wherein the post member comprises a tube portion and a plurality of legs extending distally from a distal end of the tube portion, the leg portions applying a force on the distal seal to open the aperture after medical implement is inserted into the inlet.

70. A medical valve according to claim 69, wherein the plurality of legs apply a radially directed force and a longitudinally directed force on the distal seal as the post member moves distally, the radially directed force and longitudinally directed force opening the aperture.

71. A medical valve according to claim 61, wherein at least a portion of the distal seal member inverts from a first shape to a second shape as the aperture opens.

72. A medical valve according to claim 61, wherein the valve further includes a proximal seal member with an normally closed aperture therethrough, insertion of the medical implement moving the proximal seal member distally and opening the proximal seal aperture.

73. A medical valve according to claim 72, wherein the proximal seal member is swabbable when the valve is in the closed mode.

74. A medical valve according to claim 72, wherein the first variable volume is located between the post member and the distal seal, the second variable volume being located between the post member and the proximal seal.

75. A medical valve according to claim 61, wherein the distal seal includes a protrusion extending distally from the distal seal, the normally closed aperture passing through the protrusion, the protrusion providing a dynamic back pressure seal.

76. A medical valve according to claim 61, wherein the first variable volume expands after insertion of the medical implement and the second variable volume contracts after insertion of the medical implement.
